(19) United States
(12) Patent Application Publication
(10) Pub. No.: US 2015/0201879 A1
(43) Pub. Date: Jul. 23, 2015

(54) Method and Apparatus for Diagnosing and Assessing Centralized Pain

(71) Applicant: Cerephex Corporation, Los Altos, CA (US)

(72) Inventor: Jeffrey B. Hargrove, Bancroft, MI (US)

(21) Appl. No.: 14/417,142

(22) PCT Filed: Jul. 24, 2013
(86) PCT No.: PCT/US2013/051867

§ 371 Date: Jan. 24, 2015

Related U.S. Application Data
(60) Provisional application No. 61/675,190, filed on Jul. 24, 2012.

(51) Int. Cl. A61B 5/00; A61B 5/0484; A61B 5/055

(52) U.S. Cl. CPC A61B 5/4827; A61B 5/055; A61B 5/0484; A61B 5/0053; A61B 5/0057; A61B 5/7257; A61B 5/7235; A61B 5/483

(57) ABSTRACT

Methods for central pain diagnosis and assessment, symptom severity prediction, and therapeutic intervention effect determination. The diagnosis and assessment method includes a statistical comparison between a subject's quantitative brain function assessment and either a database of quantitative assessments of brain functions of healthy individuals, or a database of quantitative assessments of brain functions of individuals known to have been suffering from chronic pain as a result of the abnormal brain function condition. Diagnosis and assessment may be accomplished using a neuroimaging device to sense and generate images representing central nervous system function, using a sensory stimulation device to stimulate brain activities associated with central sensitivity, and using a computing device to command the sensory stimulation device and neuroimaging device to test for the presence of central pain.

### TECHNICAL FIELD

The present invention relates generally to the field of diagnosing central pain disorders. More specifically, the present invention relates to methods and apparatuses for diagnosing abnormal pain processing function or mechanisms in the brain that result in central pain disorders in human subjects.

### BACKGROUND

Nociceptive pain is known to arise from stimulation of peripheral nerve endings. In response to such stimulation, a peripheral nerve ending generates a peripheral nociceptive signal that is then transmitted through the spinal cord to the brain, where it is processed through numerous pain-processing networks. Descending pathways from the brain to the spinal cord subsequently modulate pain signals, thereby increasing or decreasing pain perception.

However, it is also known that enhanced activation of central pain-processing pathways and networks, through mechanisms such as neuroplastic changes in central neuronal activity and network connectivity, can lead to spontaneous pain in the absence of peripheral nociceptive input. When this occurs, pain is said to have “centralized”, which results in lower pain thresholds, secondary hyperalgesia in uninjured areas, and sustained pain potentiation. Brain-related central pain (also known as “centralized pain”) is thought to play a prominent role in chronic pain conditions.

Central pain is generally thought of as an outcome of central sensitivity (CS), which is also known as central sensitization, central augmentation, and central hypersensitivity among other terms. CS mechanisms in the brain have been implicated in the pathology of allodynia, which is the term used to describe a condition where pain is caused by a stimulus that does not normally provoke pain. CS mechanisms in the brain have also been implicated in hyperalgesia, which is the term used to describe a condition in which pain perceived from a stimulus is greater than what would normally be expected from that stimulus. Put simply, in central sensitivity the brain magnifies painful stimuli and eventually magnifies even associated non-painful stimuli. As pointed out in Latremoliere and Woolfe (1), because CS results from changes in the properties of neurons in the central nervous system, the pain is no longer coupled, as acute nociceptive pain is, to the presence, intensity, or duration of noxious peripheral stimuli arising from neuropathic and/or inflammatory sources. Further, in chronic pain conditions the increased excitability caused by CS far outlasts the initiating noxious stimulus, that is, the nociceptive input that causes the pain to occur in the first place.

Before CS was discovered, typically only two models of pain were contemplated. The first is the aforementioned nociceptive pain model, by which specific pain pathways are activated by peripheral pain stimuli, and the amplitude and duration of the pain experienced is determined entirely by the intensity and timing of the peripheral pain inputs. The second model contemplates gate controls in the central nervous system that open and close, thus enabling or preventing pain. Medical science now recognizes CS as a third and unique model that contemplates neuroplastic changes in the functional properties and network connectivity of the central nervous system. For example, the level of resting brain activity within multiple networks (e.g. functional network connectivity and effective network connectivity) is now known to be associated with spontaneous pain in patients having central pain (2, 3). CS leads to reductions in pain threshold, increases in the magnitude and duration of responses to noxious input, and permits normally innocuous inputs to generate pain sensations. In addition, CS is also believed to be relevant in somatic symptoms associated with painful conditions, including but not limited to fatigue and sleep disorders.

The brain's role in CS is being increasingly revealed and understood in neuroscience, due in large part to the advent of functional brain imaging technologies. For example, Lee et al. (4) used functional magnetic resonance imaging (fMRI) to examine the extent to which brain activity contributes to the maintenance of CS in humans. When the intensity of pain during CS was matched to the intensity of pain during normal states, activity within the brainstem, including the mesencephalic pontine reticular formation and the anterior thalami, remained at an increased level during CS. Regarding brain areas related to the consequence of increased pain perception during CS, cortical activity, mainly in the primary somatosensory area, has been significantly correlated with the intensity of pain attributable to both the force of noxious stimulation used, and the state in which noxious stimulation was applied.

Borsook et al. (5) reviewed the literature on brain activity using neuroimaging technologies. Their review details evidence of alterations in multiple sub-cortical and cortical processing mechanisms. Those alterations include sensory, emotional/affective, cognitive, and modulatory systems that are present in chronic pain. The authors note these findings provide evidence that increases understanding of the importance of the role of numerous brain regions in the centralization of pain and the contributions of those regions to the altered brain states associated with chronic pain conditions. Similarly, Schweinhardt and Bushnell (6) review neuroimaging evidence of the active and enhanced modulatory role that the brain plays in pain processing in chronic pain patients. Schwienhardt and Bushnell also cite findings that brain activations in chronic pain involve brain circuitry not normally activated by acute nociceptive pain.

Because of this emerging understanding, the role of CS is increasingly being shown to be pathological in seemingly unrelated chronic pain conditions and syndromes including fibromyalgia, complex regional pain syndrome, phantom pain, and migraine headaches. Yunus (7) identifies no less than 14 common syndromes that lack structural pathology yet have CS as a common mechanism. These conditions further include chronic fatigue syndrome, irritable bowel syndrome, tension-type headaches, temporomandibular disorder, myofascial pain syndrome, regional soft-tissue pain syndrome, restless leg syndrome, periodic limb movements in sleep, multiple chemical sensitivity, primary dysmenorrhea, female urethral syndrome, interstitial cystitis, and post-traumatic stress disorder. Yunus also notes that CS may play a significant role in the pain associated with depression and in Gulf War Syndrome.

Giesecke et al. (8) used fMRI to demonstrate augmented central pain processing in patients with idiopathic chronic low back pain and fibromyalgia. Indeed, when equal levels of mechanical pressure intended to elicit a painful response were applied to patients and to normal controls, patients with chronic low back pain and fibromyalgia experienced significantly more pain and showed more extensive, common patterns of neuronal activation in pain-related cortical areas of the brain than did the controls. Thus, CS may play an important role in persons with chronic low back pain that persists without identifiable physical pathology.

The role of CS in persistent inflammatory conditions is also gaining recognition. In Gwilym et al. (9), fMRI illustrated significantly greater brain activation in osteoarthritis (OA) patients in response to stimulation of their referred pain areas (i.e. areas where pain persists but do not exhibit OA or related inflammation) compared with healthy controls, and the magnitude of this activation positively correlated with the extent of neuropathic-like elements to the patient's pain. The role of CS in osteoarthritis has been the subject of several other investigations (10, 11, 12). As detailed in Imamura et al. (13), the refractory, disabling pain associated with knee OA is usually treated with total knee replacement. However, a comparison of OA patients with healthy normal controls showed patients with knee OA had significantly lower pressure pain thresholds (PPT) over widespread evaluated structures beyond the knee. The lower PPT values were correlated with higher pain intensity, higher disability scores, and with poorer quality of life. This suggests that pain in these patients might be more associated with CS than with peripheral inflammation and injury. As the authors point out, the implications of the role of CS, and its potential for modulation, may provide exciting and innovative cost effective therapeutic tools to control pain, reduce disability, and improve quality of life in knee OA patients.

The diagnosis of pain generally fails to differentiate central pain processes in the brain from peripheral pain arising from an ongoing noxious stimulus. Diagnosing central pain is usually only made empirically after multiple failed therapeutic attempts reveal its likely presence. This practice results in unmet expectations for both patients and physicians, and contributes to high healthcare costs in the chronic pain clinical population. The ability to develop quantitative real-time diagnostic and assessment methods for central pain, especially methods and apparatuses making such diagnosis and assessment practical at the point-of-care, would be a significant clinical advancement. Such would improve physicians' ability to appropriately and immediately match treatments to the relevant pain mechanism, thus saving time and reducing healthcare costs.

Of relevance to the present invention, it is known that neurophysiologic information may be obtained by techniques such as electroencephalography (EEG) and fMRI. It is also known that fMRI can be used to measure neurotransmitter and neuroreceptor activity. It is also known that the analysis of numerous brain imaging and functional measures, including EEG measures (13), have been shown to produce measures related to brain networks and network connectivity that correlate to findings produced by fMRI imaging (14). Thus, the presence of brain activity associated with CS, and hence central pain, can be determined using EEG measures and analysis.

The following documents are incorporated by reference in their entirety:
“Central sensitization: a generator of pain hypersensitivity by central neural plasticity”, Latremoliere A. Woolf C J. J Pain. 2009 September; 10(9):895-926.
“Intrinsic brain connectivity in fibromyalgia is associated with chronic pain intensity”, Napadow V, LaCount L, Park K, As-Sanie S, Clauw D J, Harris R E. Arthritis Rheum. 2010 August; 62(8):2545-55.
“Disrupted functional connectivity of the pain network in fibromyalgia”, Cifre I, Sitges C, Fraiman D, Muñoz M Á, Balenzuela P, González-Roldán A, Martinez-Jauand M, Birbaumer N, Chialvo D R. Montoya P. Psychosom Med. 2012 January; 74(1):55-62.
“Identifying brain activity specifically related to the maintenance and perceptual consequence of central sensitization in humans”, Lee M C, Zambreanu L, Menon D K. Tracey I. J Neurosci. 2008 Nov. 5; 28(45):11642-9.
“A key role of the basal ganglia in pain and analgesia—insights gained through human functional imaging”, Borsook D, Upadhyay J, Chudler E H, Becerra L. Mol Pain. 2010 May 13; 6:27.
“Pain imaging in health and disease—how far have we come?”. Schweinhardt P, Bushnell M C. J Clin Invest. 2010 Nov. 1; 120(11):3788-97.
“Fibromyalgia and overlapping disorders: the unifying concept of central sensitivity syndromes”, Yunus M B. Semin Arthritis Rheum. 2007 June; 36(6):339-56.
“Evidence of augmented central pain processing in idiopathic chronic low back pain”, Giesecke T, Gracely R H, Grant M A, Nachemson A, Petzke F, Williams D A, Clauw D J. Arthritis Rheum. 2004 February; 50(2):613-23.
“Psychophysical and functional imaging evidence supporting the presence of central sensitization in a cohort of osteoarthritis patients”, Gwilym S E, Keltner J R, Warnaby C E, Carr A J, Chizh B, Chessell I, Tracey I. Arthritis Rheum. 2009 Sep. 15; 61(9):1226-34.
“Lessons from fibromyalgia: abnormal pain sensitivity in knee osteoarthritis”, Bradley L A, Kersh B C, DeBerry J J, Deutsch G, Alarcón G A, McLain D A. Novartis Found Symp. 2004; 260:258-70.
“Sensitization in patients with painful knee osteoarthritis”, Arendt-Nielsen L. Nie H, Laursen M B, Laursen B S, Madeleine P, Simonsen O H, Graven-Nielsen T. Pain. 2010 June; 149(3):573-81.
“Pain mechanisms in osteoarthritis: understanding the role of central pain and current approaches to its treatment”, Mease P J. Hanna S. Frakes E P, Altman R D. J Rheumatol. 2011 August; 38(8):1546-51.
“Impact of nervous system hyperalgesia on pain, disability, and quality of life in patients with knee osteoarthritis: a controlled analysis”. Imamura M, Imamura S T, Kaziyama H H, Targino R A, Hsing W T, de Souza L P, Cutait M M, Fregni F, Camanho G L. Arthritis Rheum. 2008 Oct. 15; 59(10):1424-31.
“Functional connectivity: the principal-component analysis of large (PET) data sets”, Friston K J. Frith C D, Liddle P F, Frackowiak R S. J Cereb Blood Flow Metab 1993; 13:5-14.
“Electrophysiological signatures of resting state networks in the human brain”, Mantini D, Perrucci M G, Del Gratta C, Romani G L, Corbetta M. Proc Natl Acad Sci USA. 2007 Aug. 7; 104(32):13170-5.
U.S. patent application Ser. No. 12/865,286 filed Jul. 29, 2010 as a 371 of PCT/US09/32639, published on Dec. 23, 2010 as Pub. No. US2010/324441 A1, and assigned to the assignee of the present application;
U.S. Provisional Patent Application Ser. No. 61/024,641, filed Jan. 30, 2008 and assigned to the assignee of the present application;
U.S. Provisional patent application Ser. No. 12/865,286, filed Jul. 29, 2010 and assigned to the assignee of the present application;
U.S. Provisional Patent Application Ser. No. 61/644,049, filed May 8, 2012 and assigned to the assignee of the present application.

### SUMMARY

A method is provided for diagnosing and assessing central pain. The method may include the steps of assessing a subject's brain function, determining the probability that a subject is suffering from chronic pain as a result of an abnormal brain function condition by obtaining a quantitative assessment of the subject's brain function, and

making a statistical comparison between the subject's quantitative brain function assessment and either a database of quantitative assessments of the brain functions of normal, healthy individuals, or a database of quantitative assessments of the brain functions of individuals known to have been suffering from chronic pain as a result of the abnormal brain function condition.

The method may alternatively include assessing a subject's brain function using a brain response test (BRT) comprising the steps of performing one or more baseline neuroimaging tests, causing one or more brain responses by applying one or more sensory stimulations, and performing one or more neuroimaging tests after the step of causing one or more brain responses. The method may also include the steps of obtaining a quantitative assessment of the subject's brain function and making a statistical comparison between the subject's quantitative brain function assessment and one or more databases of quantitative assessments of the brain functions.

A method is provided for predicting symptom severity in individuals having central pain. The method may include the steps of executing a brain response test on a subject, obtaining measures of brain activities associated with central pain in the subject by analyzing findings from the brain response testing, correlating these measures of brain activities to measures of symptom severity, creating a mathematical correlation model that provides symptom severity as a function of the measures of brain activities, and subsequently using the mathematical correlation model to predict symptom severity in individuals having central pain when measures of brain activities are known.

A method is provided for determining the effect of a therapeutic intervention in alleviating symptoms of central pain. The method may include the steps of executing a brain response test on a subject, obtaining measures of brain activities associated with central pain in the subject by analyzing findings from the brain response testing, correlating these measures of brain activities to measures of the effect of a therapeutic intervention, creating a mathematical correlation model that provides the effect of therapeutic intervention as a function of the measures of brain activities, and subsequently using the mathematical correlation model to predict the effect of therapeutic intervention in alleviating symptoms of central pain when measures of brain activities are known.

An apparatus for diagnosing and assessing central pain is provided, which may comprise a neuroimaging device that is configured to sense and generate images representing central nervous system function, a sensory stimulation device that is configured to stimulate brain activities associated with central sensitivity, and a computing device that is coupled to the neuroimaging device and the sensory stimulation device and configured to command the sensory stimulation device and neuroimaging device. The apparatus may be configured to perform a brain response test for the presence of central pain.

### BRIEF DESCRIPTION OF THE DRAWINGS

These and other features and advantages of the invention will become apparent to those skilled in the art in connection with the following detailed description and drawings, in which:

FIG. 1 is a flow chart depicting a method for diagnosing fibromyalgia;

FIG. 2 is a flow diagram of a method of diagnosing and assessing central pain;

FIG. 3 is a flow diagram of a method of predicting symptom severity in individuals having central pain;

FIG. 4 is a flow diagram of a method of determining the effect of a therapeutic intervention in alleviating symptoms of central pain; and

FIG. 5 is a schematic diagram showing an embodiment of an apparatus for diagnosing and assessing central pain.

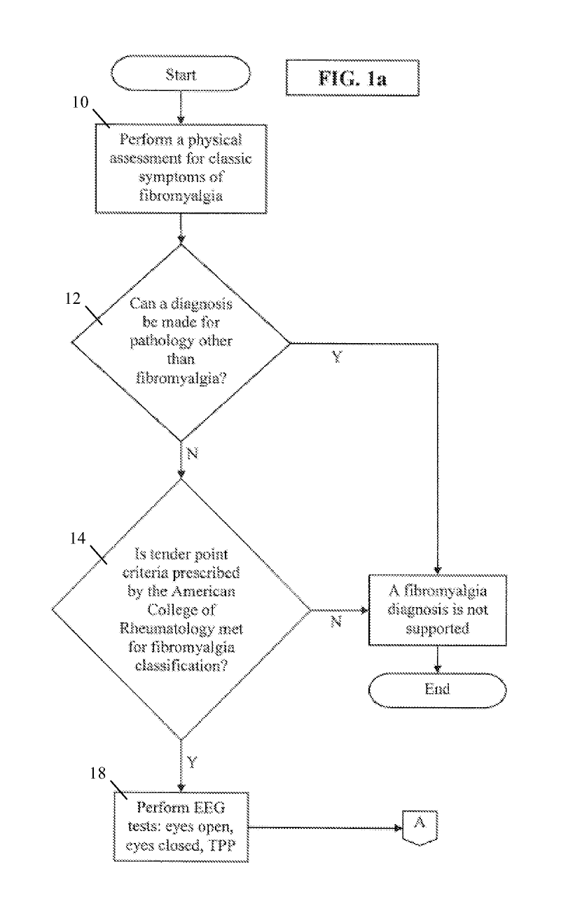

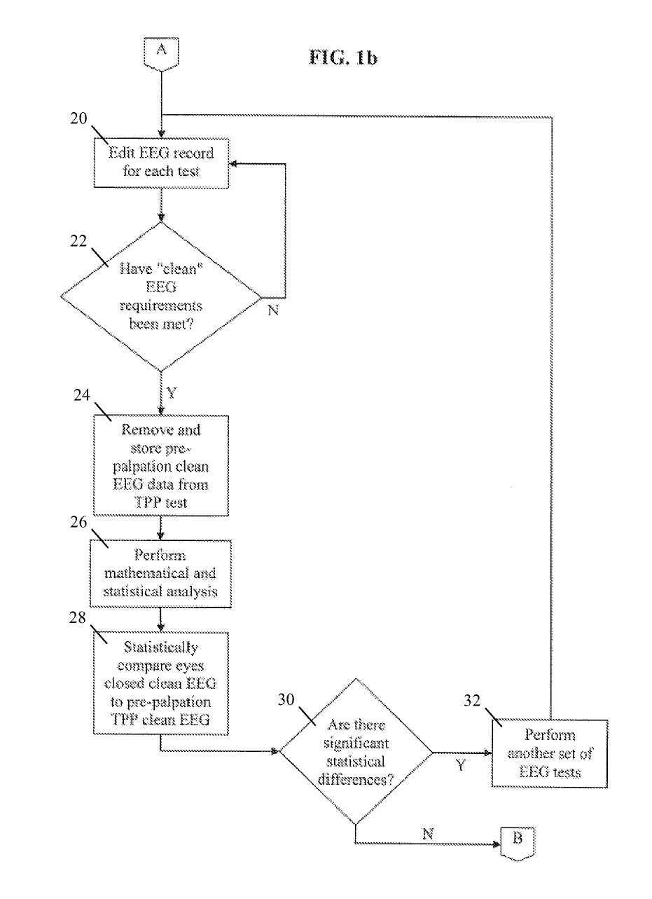

### DETAILED DESCRIPTION

In the following description of the disclosed apparatus and methods, the term “central pain”, which is also known as “centralized pain”, is intended to mean any form of pain, whether chronic or acute, that is enhanced in its characteristics; such as magnitude, duration and scope; due to abnormal brain activity associated with pain processing. Such brain activity may include, but is not limited to, central sensitivity and network connectivity.

The term “central sensitivity” is intended to mean any central nervous system condition pathologically related to hyperalgesia, allodynia, reductions in pain threshold, increases in the magnitude and duration of responses to noxious input, results in normally innocuous inputs to generate pain sensations, or results in non-painful symptoms associated with increases in central nervous system responsiveness. Central sensitivity is also known by alternate terms that include but are not limited to “central sensitization”, “central pain”, “central augmentation,” and “central hypersensitivity”.

Central sensitivity is not a manifestation or cause of an individual symptom or condition. Instead, central sensitivity results in a worsening of the effect or magnitude of one or more symptoms because of a central nervous system condition that is independent of the cause of the one or more symptoms per se. Thus, any method of treatment of central sensitivity is fundamentally different from treatment of a specific symptom. For example, treatment of pain augmentation by central sensitivity is inherently different than treatment of pain under traditional nociceptive models of pain.

The terms “network connections” and “network connectivity” are intended to mean various forms of relationships between brain regions involved in processing of information such as pain. For example, “functional connectivity” refers to a statistical correlation between the activities of different brain regions. “Effective connectivity” denotes not simply a statistical but a causal influence between two brain regions.

The term “alleviate” or “alleviating” is intended to mean the act of reducing, making less severe, mitigating, treating, or eliminating a condition and/or its symptoms for any period of time.

Except where the context requires otherwise, the term “comprise” and variations of the term, such as “comprising”, “comprises” and “comprised” are not intended to be exclusive. Where, for example, a form of the word “comprise” is used to refer to one or more additives, components, integers or steps; its use is not intended to exclude other additives, components, integers or steps.

Where the terms “integral” or “integrated” are used to describe a relationship between two or more elements, the terms are intended to indicate that such elements are joined together in a manner that does not allow separation of elements from one another without diminishing or destroying a function of one or more of the elements.

The term “stimulation signal” is intended to mean any energy signal used in the process of stimulating a tissue such as a brain by transmitting an energy signal generated by a device such as an electrical stimulator, or a magnetic stimulator such as a transcranial magnetic stimulator. Other terms used to refer to such a signal may include but are not limited to “cortical stimulation”, “neuromodulation” and “neurostimulation”.

The term “neuroimaging test” is intended to mean any medical test that provides visual indication, measures, or other data that can be used to make an assessment about central nervous system function, including brain function. Types of tests that the term “neuroimaging test” may be used to refer to include, but are not limited to, magnetic resonance imaging, computer aided tomography, positron emission tomography, or single photon emission computed tomography, and may also include brain electrical function tests such as electroencephalography or magnetoencephalography.

The term “brain activities” is intended to refer to any brain activities that are known in the art to be associated with central sensitivity. Such brain activities are intended to include, but are not limited to, abnormal condition, abnormal function, abnormal response, abnormal regions of activation, abnormal network connectivity, abnormal release of neurochemicals, abnormal uptake of neurochemicals, abnormal electrical activity, or abnormal metabolism.

The term “brain function” is intended to mean any action or process of a brain that is within the brain's normal state of operation.

The term “spectral segments” is intended to mean frequency components of an electrical signal that includes individual frequency components, and in the case of an EEG signal, that includes groupings of frequency components commonly known as “frequency bands”, such bands including, but not limited to the “delta” band (nominally 1-3.5 hertz), the “theta” band (nominally 4-7.5 hertz), the “alpha” band (nominally 8-12 hertz) and the “beta” band (nominally 12.5-25 hertz).

The term “resting EEG” is intended to mean electroencephalogram signals that are collected with the subject's eyes either open or closed and during periods of no significant physical activity, mental activity, or any other form of engagement that may cause the brain to be stimulated significantly or engaged in elevated brain function.

A method is provided for diagnosing and assessing a brain-related chronic pain disorder. The method includes assessing a human subject's brain function and then determining the probability that the subject is suffering from chronic pain related to an abnormal brain function condition by obtaining a quantitative assessment of the subject's brain function and making a statistical comparison between the subject's quantitative brain function assessment and a database of quantitative assessments of the brain functions of individuals known to have been suffering from chronic pain as a result of the abnormal brain function condition. The assessment of a subject's brain function may include obtaining an electroencephalogram (EEG) of the subject's electrical brain activity, and the determination of the probability that the subject is suffering from chronic pain as a result of an abnormal brain function condition may include determining the probability that the subject is suffering from a chronic pain condition such as fibromyalgia by obtaining a quantitative assessment of the subject's EEG (qEEG) and making a statistical comparison between the subject's qEEG and a database of qEEGs of individuals known to have been suffering from fibromyalgia.

A physical assessment may first be performed of a human subject presenting with a complaint of symptoms characteristic of a chronic pain condition such as fibromyalgia. The physical assessment may include, among other things, a determination of chronic widespread pain, sleep difficulty, fatigue, morning stiffness of the muscles and joints, cognitive difficulty and other symptoms associated with the condition. Where, for example, fibromyalgia is suspected, the physical assessment may also include tests performed to exclude various non-fibromyalgia conditions as the cause of the symptoms. Such further testing may include palpation of 18 tender points in the manner prescribed by the American College of Rheumatology (ACR), with such palpation being performed to determine whether the subject has an abnormal sensitivity to pain. Where, for example, idiopathic chronic low back pain ICLBP) is suspected, the physical assessment may include tests performed to exclude various non-ICLBP conditions as the cause of the symptoms. Such further testing may include palpation of tender points other than the 18 tender points prescribed by the ACR and/or may include physical tests other than tender point palpation.

In the absence of a definitive diagnosis, an EEG test may be performed in addition to the physical assessment. Specifically, the subject may be made comfortable by, for example, being seated or reclined. Preparation of the scalp in accordance with commonly followed procedures for performing a clinical EEG may be done by a person of sufficient competence. EEG electrodes may then be adapted to be worn on the scalp, preferably in scalp locations identified as the “International 10-20” standard sites, using common methods of affixing the electrodes such that they rest on or otherwise contact tissues.

While any number of electrodes may be used, a preferred number is either 19 or 24, in accordance with the number of electrode sites used to construct various independent databases utilized to represent the EEG of a healthy normal population.

Records of the subject's EEG from each electrode site may then acquired under the conditions of both their eyes being closed and their eyes being open, with each condition producing a separate data record. In other words, an “eyes open” EEG record may be obtained, which includes EEG data obtained from each electrode site while the subject's eyes are open and an “eyes closed” EEG record may be obtained, which includes EEG data obtained from each electrode site while the subject's eyes are closed. Preferably, a minimum of five minutes of EEG data may be obtained from each electrode site for each “eyes open” EEG record and a minimum of five minutes of EEG data may be obtained from each electrode site for each “eyes closed” EEG record to assure that enough EEG data is recorded to produce statistically significant samples from each electrode site, both with the subject's eyes open and with the subject's eyes closed. This is further described below.

Preferably, an additional test may be performed in which at least one additional EEG record is made that includes EEG data obtained at each electrode site while pain is elicited in the subject. In diagnosing or assessing conditions such as fibromyalgia, a number of tender points on the subject's body may be palpated. In this test, henceforth referred to as a “tender point palpation (TPP) test”, a number of tender points on the subject's body, preferably ranging between one and 18 when diagnosing or assessing fibromyalgia, are identified and serially palpated, preferably with an algometer. Preferably, four tender points may be chosen, and, preferably, those four points include tender points adjacent the right and left lateral epicondyle of the arms, and tender points adjacent the right and left costochondral junctions of the second rib. While the subject's eyes are preferably closed during this test, it should not be confused with the “eyes closed” test described above.

The TPP test may be executed by acquiring an EEG record (“TPP” EEG record) including EEG data obtained from the electrode sites for a first tender point by first commencing the acquisition of EEG data and then, a short period of time later, commencing palpation of the first tender point. Preferably, the period of time between the commencement of data acquisition and the commencement of palpation of the first tender point may be between one and three hundred seconds. Palpation of the first tender point may be accomplished by pressing on the tender point with an algometer, preferably at a rate of approximately one kilogram per centimeter squared per second, until the subject reports a painful sensation. Preferably, palpation pressure may be removed as soon as the subject reports a painful sensation. A record is made of the amount of the pressure being applied at the moment the subject reports a painful sensation. Although the TPP EEG record may be obtained while the subject's eyes are closed, it should not be confused with the “eyes closed” EEG record described above.

Further according to the TPP test method, the acquisition of the TPP EEG record may include continued recording of EEG data (with the subject's eyes closed) for a period of time after release of palpation pressure, preferably between 1 and 300 seconds, and most preferably, for at least 60 seconds. A comparison may then be made between EEG data collected before application of palpation pressure and EEG data collected after release of palpation pressure. This comparison may then be used to make diagnostic findings. Such findings may include changes in brain EEG activity, when comparing EEG after release of palpation pressure to EEG before palpation pressure, in specific regions of the brain characteristic of a brain-related chronic pain condition, but not otherwise anticipated in a healthy normal individual.

Following this period, a second and subsequent tender point may be serially palpated, preferably with an algometer, in the same manner as described for the first, with TPP EEG records being recorded for each by recording the eyes closed EEG for each site in the manner described with regard to obtaining the TPP EEG record for the first site. This process may be repeated for each chosen tender point. Accordingly, the resulting EEG data record includes the TPP EEG records acquired for each chosen tender point.

The “TPP” EEG records may be acquired for a period of time that is sufficient to extract from each “TPP” EEG record a minimum of 60 seconds of “clean” EEG data, that is, data free of extraneous electrical noise such as that from electromyographic movement. Preferably, all EEG records (“eyes open” EEG records, “eyes closed” EEG records, and “TPP” EEG records) may be individually edited to provide from each EEG record a minimum of 60 seconds of clean EEG. Preferably, the clean data is obtained so as to present a high degree of statistical consistency. Such measures as “Split-Half” reliability, which is the ratio of variance between the even and odd seconds of the time series of selected clean EEG; and “Test Re-test” reliability, which is the ratio of variance between the first half and the second half of the selected clean EEG segments may be used. Preferably, clean EEG data is obtained such that measures of these ratios are a minimum of 0.95 and 0.90 respectively, which is consistent with levels of reliability commonly published in EEG literature.

With regard to the TPP test method, clean data includes that EEG data acquired after palpation of a tender point, and does not include any EEG data acquired during the palpation of a tender point. In addition, to assess the stability of a TPP EEG record, EEG data acquired before palpation of a tender point may be removed, edited and statistically compared to like data in the “eyes closed” EEG record obtained from the eyes closed EEG test. Stability of the “closed eyes” and TPP EEG records is indicated by a finding that there is no statistically significant difference between the “eyes closed” EEG record and the pre-palpation portion of the. TPP EEG record. A contrary finding indicates instability and a need to repeat the EEG tests.

Further to the method, and in the preferred embodiment, clean “eyes open”, “eyes closed”, and “PPT” EEG records may be then mathematically analyzed for various time domain and frequency domain parameters of their respective electrical signals. These analyses may include, but are not limited to voltage and current analyses, frequency spectrum analyses using methods such as a Fast Fourier Transform or wavelet analysis, an absolute power analysis, a relative power analysis, a phase analysis, a coherence analysis, an amplitude asymmetry analysis, and localization of electrical activity in the brain using inverse EEG computation analysis.

Findings from the aforementioned analyses may then be statistically compared to the same parameters determined from “eyes open”, “eyes closed”, and “PPT” EEG records taken from an age and gender matched database of healthy normal individuals. Such statistical analyses may include, but are not limited to deviations from a standard normal distribution. Findings of statistically significant abnormal deviation, or lack thereof; may then be presented in a graphical or numerical format for analysis by a competent health care professional or person of similar expertise.

EEG abnormalities consistent with those observed in a sample population of fibromyalgia patients may include, but are not limited to one or more of the following: (1) an overall reduction in EEG power across all spectra in either of the eyes open or eyes closed conditions; (2) statistically significant low EEG power levels in frontal or temporal regions of any of the delta (1-3.5 hertz), theta (4-7.5 hertz) or alpha (8-12 hertz) frequency segments of EEG for the eyes closed condition; (3) statistically significant low coherence among the frontal EEG sites for the delta or theta EEG segments in either of the eyes closed or eyes open conditions; (4) statistically significant high relative beta (12.5-25 hertz) absolute power in the parietal region of the brain for either of the eyes closed or eyes open conditions. The magnitude of statistical variation considered to be statistically “significant” may vary depending on the application. For example, in research, a difference between a sample and a population measure generally has to have a p-value of 0.01 or less for the difference to be considered statistically “significant”. However, in clinical application statistically significant differences may be declared with p-values at the 0.1 level or less.

Further EEG abnormalities consistent with those observed in a sample population of fibromyalgia patients, and drawn particularly to the TPP test method, may include but are not limited to a finding of (1) a statistically significant increase in EEG absolute power, particularly in the alpha and beta segments, in the parietal and occipital areas of the brain as compared to the “eyes closed” EEG record (“eyes closed” EEG findings without tender point palpation) for the same subject; or (2) a statistically significant increase in coherence in the alpha or beta segment of EEG.

A diagnosis of fibromyalgia may be made when physical assessment findings that support a diagnosis of fibromyalgia are augmented by (1) at least one abnormal finding resulting from the TPP test, preferably a finding of a statistically significant increase in EEG absolute power, and particularly in the alpha and beta segments, in the parietal and occipital areas of the brain as compared to the eyes closed findings without tender point palpation for the same subject; and preferably (2) at least one abnormal finding resulting from the eyes closed EEG test, preferably statistically significant low EEG power levels in frontal or temporal regions of any of the delta, theta or alpha frequency segments of EEG for the eyes closed condition, and most preferably with an additional finding of statistically significant low coherence among the frontal EEG sites for the delta or theta EEG segments.

Clean EEG records from a subject may be mathematically analyzed for various time domain and frequency domain parameters of their electrical signals, consistent with analysis techniques already described, and then findings from these mathematical analyses may be statistically compared to like parameters taken from an age and gender matched database of individuals known to have fibromyalgia. The statistical comparisons may include, but are not limited to deviations from a standard normal distribution of like EEG measures associated with members of a database of individuals known to have fibromyalgia. The results of those comparisons may then be presented in a graphical or numerical format for analysis by a competent health care professional or person of similar expertise for the existence of statistically significant abnormal deviations, or the lack thereof. A finding in support of a fibromyalgia diagnosis would be supported if there is an absence of any significant deviation between measures from a subject's clean EEG and those from a database comprising individuals known to have fibromyalgia.

Similarly, clean EEG from a subject may be mathematically analyzed for various time domain and frequency domain parameters of its electrical signals, consistent with analysis techniques already described, and then findings from these mathematical analyses may be statistically compared to like parameters determined from an age and gender matched database of individuals known to have a chronic pain condition other than fibromyalgia.

The statistical comparisons may include, but are not limited to deviations from a standard normal distribution of like EEG measures associated with members of a database of individuals known to have the chronic pain condition. The results of those comparisons may then be presented in a graphical or numerical format for analysis by a competent health care professional or person of similar expertise for the existence of statistically significant abnormal deviations, or the lack thereof. A finding in support of a chronic pain condition diagnosis would be supported if there is an absence of any significant deviation between measures from a subject's clean EEG and those from a database comprising individuals known to have the chronic pain condition.

To determine the probability that a subject belongs to a population of individuals suffering from fibromyalgia a statistical comparison may be made of EEG parameters of the subject, as determined from the aforementioned analyses, to like EEG parameters determined from a database of individuals known to suffer from fibromyalgia. The statistical comparison may include, but is not limited to, determination of z-statistics associated with specific EEG measures from a standard normal distribution determined from the database of individuals known to suffer from fibromyalgia. Probability of inclusion in the population of individuals suffering from fibromyalgia would result from findings that subject measures cannot be excluded from the database standard normal distribution. Assuming that the data in the database of fibromyalgia patient EEG is normally distributed, then statistics such as the t-statistic or the z-statistic can be used to determine the probability that the sample EEG belongs to the population of fibromyalgia sufferers. If the probability is sufficiently low (e.g. p<0.01) then a conclusion could be made that the sample does not belong to that population.

Similarly, the probability that a subject belongs to the population of individuals suffering from a chronic pain condition other than fibromyalgia may be determined by making statistical comparison of EEG parameters of a subject, determined from the aforementioned analyses, to like EEG parameters determined from a database of individuals known to suffer from that chronic pain condition. The statistical comparison may include, but is not limited to, determination of z-statistics associated with specific EEG measures from a standard normal distribution determined from the database of individuals known to suffer from the chronic pain condition. Probability of inclusion in the population of individuals suffering from the chronic pain condition other than fibromyalgia would result from findings that subject measures cannot be excluded from the database standard normal distribution.

In addition, findings from aforementioned analyses of clean EEG records from a subject may be statistically correlated to measures of symptom severity. As previously described, analysis findings may be mathematically analyzed for various time domain and frequency domain parameters of their electrical signals. A number of measures of the magnitude of deviation from standard normal distributions of either healthy normal EEG, known fibromyalgia patient EEG, or from EEG of individuals known to suffer from a chronic pain condition other than fibromyalgia can be determined. The magnitudes are presumed to be related to the severity of the condition, and may be statistically correlated to such symptom measures that may include, but are not limited to tender point pain pressure thresholds as determined by an algometer, and various other indices of pain derived from the algometry measures (e.g. the sum of all 18 tender point pain tolerance measures, the average of all 18 tender point pain tolerance measures, etc.). Such analysis has utility in both predicting symptom severity in individuals with fibromyalgia, and in determining the effect of therapeutic intervention to correct or manage symptoms of fibromyalgia.

Also the above-described EEG testing and statistical analysis methods may be repeated on a subject following a period of therapeutic intervention on the subject. The results of these statistical analyses may be statistically compared to like statistical analyses of the subject accomplished before therapeutic intervention was started. This comparison might include, but is not be limited to, paired t-testing statistics, correlation analysis of changes in symptom severity, and subsequent comparison to a database of age and gender matched healthy normal individuals. The comparisons could be used as a means of assessing the effectiveness of a chosen therapeutic intervention, or as a means of determining if an alternate intervention may be indicated in the absence of treatment effect from a current therapeutic intervention. The comparisons could also be used as a means of determining if further therapeutic intervention may be indicated in the absence of any abnormal findings. With regard to the TPP test, repeat testing may include applying tender point pressure with an algometer only to the levels required to cause a painful response recorded in the same testing performed before therapeutic intervention.

EEG data may be acquired from a subject at a first location (e.g. a clinical location) and the EEG data may be transferred via electronic means to another location (e.g. a central analysis location) for the herein described analysis and statistical comparisons. The electronic means of data transfer may include, but is not be limited to, data transfer across a local area network or the internet. Analyses and statistical findings may then be transferred from the central analysis location to the clinical location where they can be used in various ways by a physician or similarly qualified health care professional for the determination of best clinical practice and therapeutic intervention.

EEG data may also be acquired from a subject at a first location (e.g. a clinical location) and the EEG data transferred via electronic means to another location (e.g. a central analysis location) for the purpose of increasing the size of various databases of individuals known to be suffering from fibromyalgia, individuals known to be suffering from a chronic pain condition other than fibromyalgia, and/or healthy normal individuals.

In testing for chronic pain conditions other than fibromyalgia, other, more general physical tests may be performed. Some of those tests may include a form of tender point palpation that differs from that typically done in testing for fibromyalgia, and that differs in a way that makes the testing more useful in diagnosing other chronic pain conditions. For example, tests involving algometer palpation may be performed at several points on the body of a suspected ICLBP patient, but not necessarily at the same 18 tender points described above for diagnosing and/or assessing fibromyalgia. Testing for ICLBP may include some other form of tender point palpation including physical action that causes reproduction of the back pain. Just as in the method disclosed for diagnosing and/or assessing fibromyalgia, this general physical test may be done following a period of EEG collection, and then additional EEG data may be captured after the test. Further, just as in the method disclosed for diagnosing and/or assessing fibromyalgia, differences in the EEG data may then be analyzed and/or statistically compared to determine if the result belongs to a particular chronic pain condition such as ICLBP. For example, the ideal test for an ICLBP patient might include palpation of four FM tender points and performance of a number of other physical actions that cause reproduction of pain specific to ICLBP patients. If the EEG analysis then shows a negative finding for the fibromyalgia tender points but a positive finding for the back pain actions, then a conclusion that the patient has ICLBP would be supported rather than a conclusion that the patient is suffering from fibromyalgia.

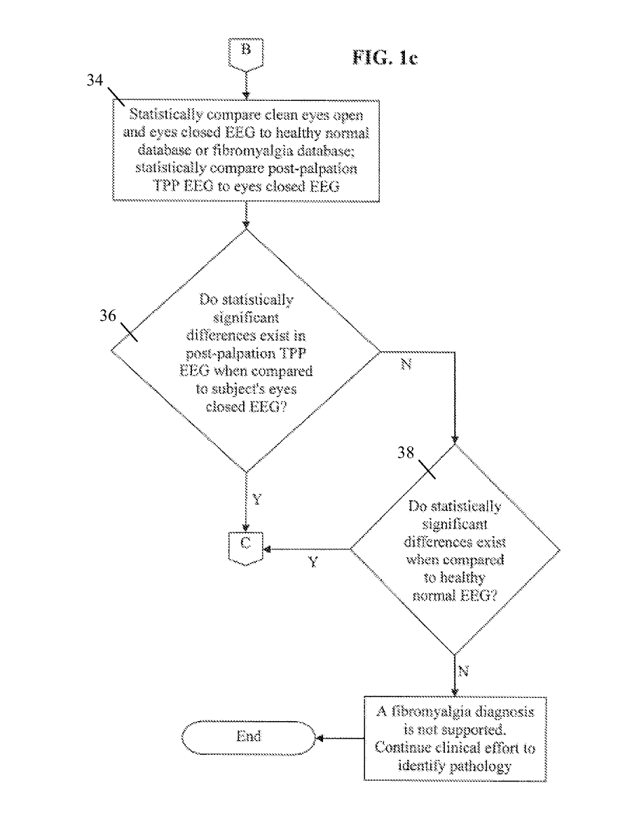

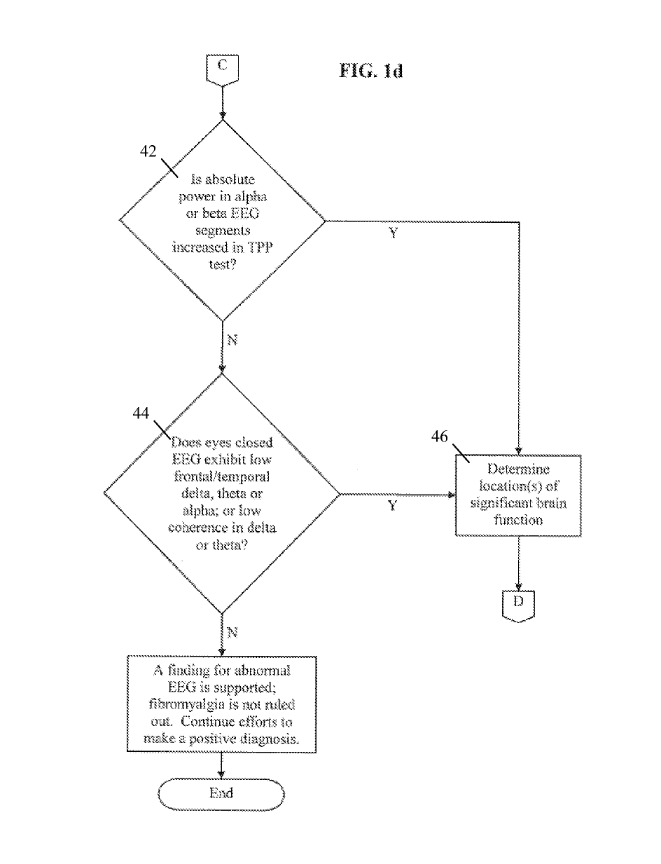

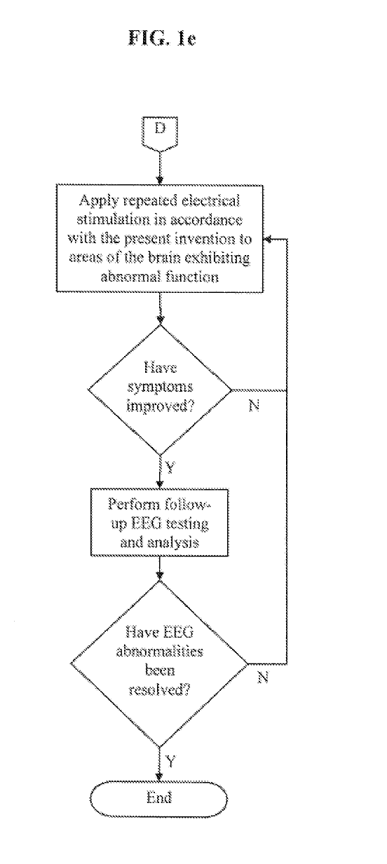

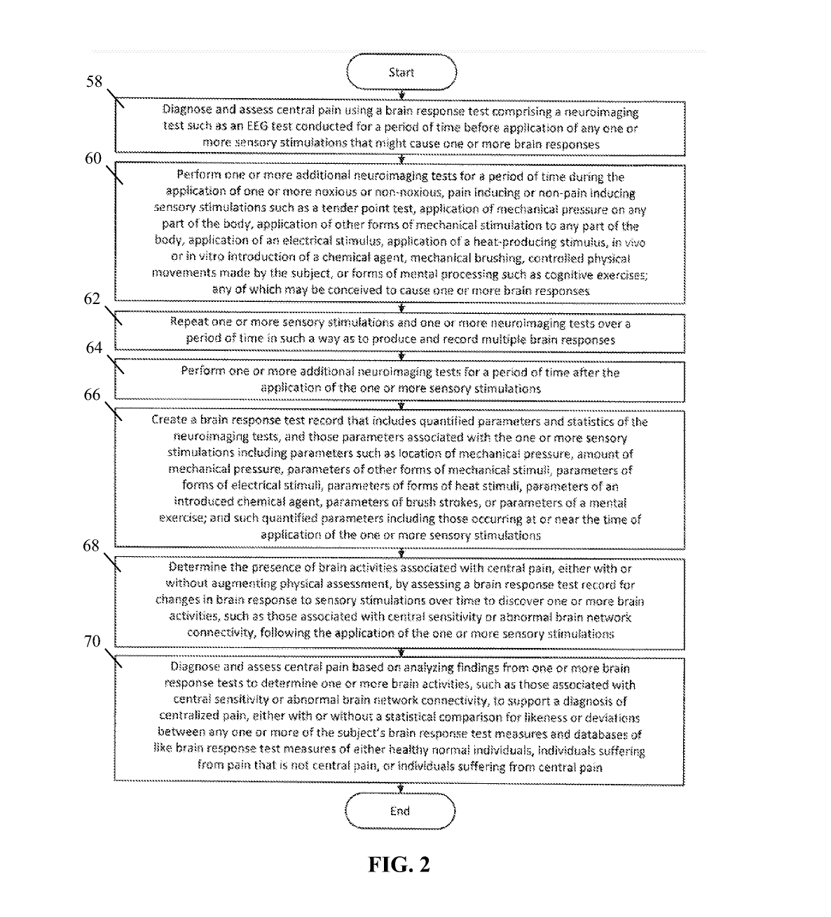

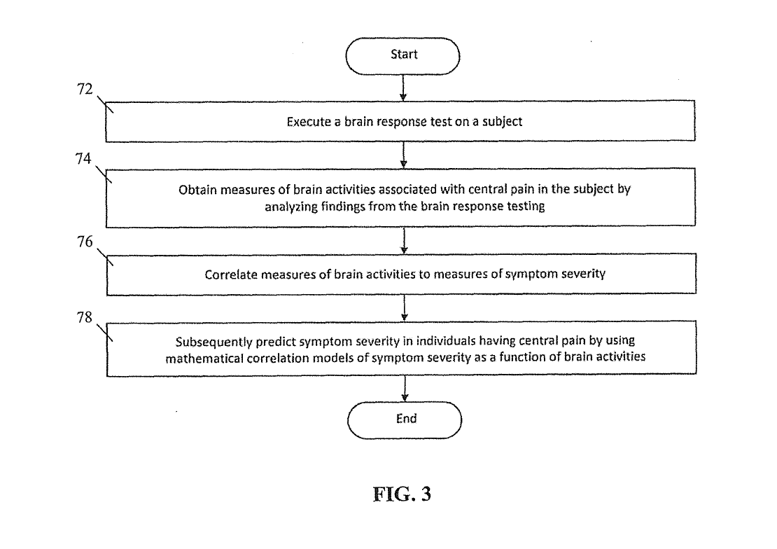

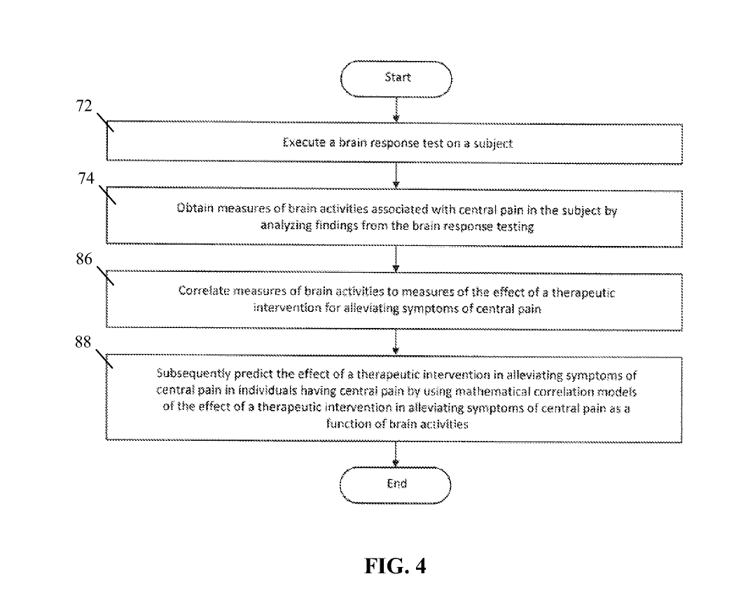

With reference to FIGS. 2-4, a method is provided for diagnosing or assessing central pain such as that arising from abnormal brain function, including but not limited to, central sensitivity and abnormal network connectivity involved in pain processing. The method includes assessing a human subject's brain function and then determining the probability that the subject is suffering from central pain by obtaining a quantitative assessment of the subject's brain function and making a statistical comparison between the subject's quantitative brain function assessment and one or more databases of quantitative assessments of the brain functions, such as a database of individuals known to be suffering from central pain, a database of individuals known to be suffering from pain that is not central pain, or a database of healthy normal individuals.

The assessment of a subject's brain function may include obtaining a neuroimaging test such as, but not limited to, magnetic resonance imaging, computer aided tomography, positron emission tomography, or single photon emission computed tomography, and may also include brain electrical function tests such as electroencephalography or magnetoencephalography. The determination of the probability that the subject is suffering from central pain may include determining the probability that the subject is suffering from central pain by obtaining a quantitative assessment of the subject's neuroimaging test and making a statistical comparison between the quantitative assessment and a database of like quantitative assessments of healthy individuals, individuals known to be suffering from central pain, or individuals suffering from pain that is not central pain.

A physical assessment may first be performed of a human subject presenting with a complaint of symptoms characteristic of central pain, such as chronic pain with no clear etiology. The physical assessment may include, among other things, a determination of chronic widespread pain, sleep difficulty, fatigue, cognitive difficulty and other symptoms associated with abnormal brain function involved in central pain. In the absence of a definitive diagnosis, a diagnostic or assessment test for central pain may be performed in accordance with the present invention.

The method may include the step of assessing the brain function of a subject to determine the presence of central pain, whereas the step includes, but is not limited to, making measures of a brain activities, e.g., of a brain function, brain conditions or brain anatomy, either by direct assessment techniques known in the art such as neuroimaging, or by indirect assessment such as analysis of other biological measures. The assessment step includes use of any method known in the art to determine the presence of any brain activity known to be associated with central pain, including but not limited to, central sensitivity or abnormal levels of network connectivity. One skilled in the art of medical assessment may administer and interpret one or more assessments designed to detect central pain. Such assessments may include any one or more known neuroimaging tests. Such assessments may also be used for detecting the presence and identifying the location of one or more abnormal brain activities through interpretation.

In a preferred embodiment, a means of assessing a brain to determine the presence of central pain in a subject includes the use of one or more neuroimaging tests utilizing methods and apparatuses known in the art, with the neuroimaging tests being performed before, during and after the application of any one or more forms of sensory stimulation (SS) intended to cause a brain response. A neuroimaging test performed before an SS is henceforth referred to as a “baseline” neuroimaging test. A neuroimaging test performed after an SS is henceforth referred to as a “post-SS” neuroimaging test. The combination of neuroimaging tests and application of one or more sensory stimulations is henceforth referred to as a “brain response test” (BRT). The SS includes any noxious, pain inducing or non-painful means. In a preferred embodiment, a BRT may include an electroencephalogram (EEG) test performed with eyes closed or eyes open, with at least one additional EEG record made that includes EEG data obtained during and after the application of any one or more forms of an SS.

One embodiment of an SS is palpation of tender points on the subject's body, consistent with the method described herein as a tender point test. Other means of causing a painful or noxious SS for the purposes of a BRT may include, but are not limited to, application of mechanical pressure on any part of the body, application of other forms of mechanical stimulation to any part of the body (e.g. a “pinch”), application of an electrical stimulus, application of a heat-producing stimulus, and in vivo or in vitro introduction of a chemical agent meant to elicit a painful or non-painful response. Means of causing a non-painful SS for the purposes of a BRT may include, but are not limited to, forms of typically non-painful physical contact including mechanical brushing, controlled physical movements made by the subject, and various forms of mental processing such as cognitive exercises.

Further to the application of an SS, the method includes any number of applications of stimulation to elicit any number of brain responses. For example, a single SS may be applied to produce a single brain response. Alternately, a series of SS applications may be made over a period of time to produce multiple brain responses so that a BRT may include assessing changes in brain response over time. Such series of PS applications may include one or more applications of any combination of noxious, painful or non-painful stimuli, with a period of rest between each application ranging from one second to several minutes. Such assessment of changes in brain response may include, but are not limited to, quantification of temporal summation of pain, also known in the pain literature as “wind up”.

The BRT test may be executed by acquiring a brain response record using any means of neuroimaging test. In a preferred embodiment, a brain response test EEG (“BRT EEG”) record is obtained that includes EEG data obtained for a period of time before, during, and after the application of any number of an SS. EEG data may be obtained from EEG electrode sites for a period of time, preferably ranging from one second to 15 minutes, prior to commencement of a first SS. During application of an SS, EEG data obtained may be denoted as EEG collected during application of the SS. Data collected during application of an SS may have unwanted aspects. For example, EEG data collected during the application of an SS may also contain measurements of electromyographic signals arising from muscle contractions a patient may make as a result of feeling a sensation such as pain. Accordingly, the data collected during the application of an SS may or may not be removed in subsequent analysis according to the method. Further to the embodiment, EEG data may be obtained from EEG electrode sites for a period of time, preferably ranging from one to 15 minutes, after application of an SS.

Further to the BRT test method, a record is made quantifying parameters associated with the one or more SS being used. For example, if an SS involves palpation of a tender point, then the location and amount of mechanical pressure being applied at or near the time the subject reports a painful sensation may be recorded. Other examples of quantification of an SS may include, but are not limited to, the amount of pressure on any body part required to elicit pain, parameters of other forms of mechanical stimuli, parameters of forms of electrical stimuli, parameters of forms of heat stimuli, parameters of an introduced chemical agent, parameters of brush strokes and parameters of a mental exercise.

Further to the BRT test method, the recording of EEG may continue for a period of time after completion of each of the one more SS applications, including a final SS application, with the period of time preferably being between one second and 15 minutes. The process of application of an SS and subsequent recording of EEG may be repeated until all intended applications of an SS are completed. Accordingly, the resulting EEG data record includes the BRT EEG records for all applications of SS.

The BRT EEG records may be acquired for a period of time that is sufficient to extract from each BRT EEG record a record of “clean” EEG data, that is, EEG data that have minimal non-EEG signals such as extraneous electrical noise arising from, for example, instrumentation anomalies or electromyographic movement. Preferably, a record of clean EEG data is sufficient to provide enough EEG data to perform any one of a number of EEG analyses known in the art with a sufficiently high degree of statistical confidence. More preferably, all EEG records according to the method may be individually edited to provide from each EEG record a period comprising a minimum of 60 seconds of clean EEG. With regard to the BRT test method, clean data preferably does not include any EEG data acquired during the application of an SS.

Further to the BRT test method, and in the preferred embodiment, clean EEG records may be then mathematically analyzed for various time domain and frequency domain parameters of their respective electrical signals. These analyses may include, but are not limited to voltage analysis, current analysis, voltage and current analysis, frequency spectrum analysis using Fast Fourier Transform (FFT) analysis, frequency spectrum analysis using a wavelet analysis method, frequency spectrum analysis using absolute power analysis method, frequency spectrum analysis using relative power analysis method, frequency spectrum analysis using phase analysis method, frequency spectrum analysis using coherence analysis method, frequency spectrum analysis using amplitude symmetry analysis method, phase analysis, various forms of network analysis and source localization of electrical activity in the brain using inverse EEG computation analysis. The purpose of such analyses is to determine the presence of one or more abnormal brain activities, e.g., brain function, brain condition, brain anatomy or related brain measures that indicate central pain such as, but not limited to, central sensitivity and abnormal levels of network connectivity.

According to the BRT test method, a finding of central pain is made by analyzing findings from the aforementioned BRT analyses. Such findings may include, but are not limited to, a determination of a brain activity associated with central sensitivity or abnormal brain network connectivity associated with pain processing. In a preferred embodiment, BRT EEG records may be statistically compared to the same parameters determined from EEG records taken from age and gender matched databases of either healthy normal individuals or individuals that are suffering from pain that is not central pain. Such statistical analyses may include, but are not limited to deviations from a standard normal distribution. Findings of statistically significant abnormal deviation, or lack thereof, may then be presented in a graphical or numerical format for analysis by a competent health care professional or person of similar expertise.

EEG abnormalities consistent with subjects suffering from central pain may include measures indicative of central sensitivity or abnormal network connectivity including, but not limited to one or more of the following: (1) abnormal levels of in EEG power in spectral segments of resting EEG measures, including but not limited to, an abnormal level of EEG power across the entire resting EEG spectra; (2) abnormal levels of coherence or phase shift between at least two resting EEG sites; (3) abnormal levels of resting EEG relative power in particular regions of the brain.

Further EEG abnormalities consistent with subjects suffering from central pain, and drawn particularly to the EEG BRT test method, may include but are not limited to a finding of (1) statistically significant increases in EEG absolute power, particularly in the alpha and beta segments, in the parietal, occipital, and temporal areas of the brain as compared to the resting EEG record for the same subject; or (2) statistically significant increases in coherence in spectral segments of the BRT EEG record as compared to the resting EEG record for the same subject.

A determination of central pain may be made when physical assessment findings that support a diagnosis of central pain are augmented by assessing a brain following a BRT. The assessment of a BRT may include a statistical comparison between any one or more of the subject's BRT measures and a database of like BRT measures of either healthy normal individuals, individuals suffering from pain that is not central pain, or individuals suffering from central pain. Alternately, central pain may be diagnosed by statistically determining one or more deviations between a subject's one or more BRT measures and like BRT measures obtained from at least one healthy normal individual or at least one individual suffering from pain that is not central pain; then comparing the one or more deviations to like deviations detected in a sample population of subjects known to be suffering from central pain.

In a preferred embodiment, clean resting EEG or BRT EEG records from a subject may be mathematically analyzed for various time domain and frequency domain parameters of their electrical signals, consistent with analysis techniques already described, and then findings from these mathematical analyses may be statistically compared to like parameters taken from age and gender matched databases of either healthy normal individuals, individuals suffering from pain that is not central pain, or individuals known to be suffering from central pain. The statistical comparisons may include, but are not limited to deviations from a standard normal distribution of like EEG measures associated with members of databases of healthy normal individuals, individuals suffering from pain that is not central pain, or individuals known to be suffering from central pain. The results of those comparisons may then be presented in a graphical or numerical format for analysis by a competent health care professional or person of similar expertise for the existence of statistically significant abnormal deviations, or the lack thereof. A central pain diagnosis would be supported if one or more findings of either resting EEG or BRT EEG records are consistent with like findings from a database comprising individuals known to be suffering from central pain. More preferably, a central pain diagnosis would be supported if one or more findings of either resting EEG or BRT EEG records are consistent with statistical significance to like findings from a database comprising individuals known to be suffering from central pain.

Further according to the BRT test method, measures of an abnormal brain activity, e.g., brain function, brain condition, brain anatomy or related brain measures arising from analyses of BRT test findings from a subject may be correlated to measures of symptom severity, such as but not limited to pain severity. Such correlation may be used to create mathematical correlation models such as a mathematical model that provides for symptom severity as a function of measures of brain activities, or such as the effect of a therapeutic intervention as a function of measures of brain activities. Such mathematical correlation models may subsequently be used to predict symptom severity in individuals having central pain, or to determine the effect of a therapeutic intervention to alleviate symptoms of central pain, when measures of brain activities are known.

Further, BRT test analyses according to the method may also be used for determining the location of abnormal brain activity and further for determining points for application of therapeutic methods for alleviating central pain, including but not limited to, cortical stimulation methods.

Further, BRT testing and statistical analysis methods may be repeated on a subject following a period of therapeutic intervention on the subject for alleviating central pain, including but not limited to, cortical stimulation methods. The results of these repeat statistical analyses may be statistically compared to like statistical analyses of the subject accomplished by performing BRT testing before therapeutic intervention was started. This comparison might include, but is not be limited to, paired t-testing statistics, correlation analysis of changes in symptom severity, and subsequent comparison to databases of either healthy normal individuals, individuals suffering from pain that is not central pain, or individuals known to be suffering from central pain. The comparisons could be used as a means of assessing the effectiveness of a chosen therapeutic intervention, or as a means of determining if an alternate intervention may be indicated in the absence of treatment effect from a current therapeutic intervention. The comparisons could also be used as a means of determining if further therapeutic intervention may be indicated in the absence of any abnormal findings. With regard to the BRT test, repeat testing may include the application of one or more SS forms. The application of the one or more SS forms may be done in accordance with types and parameters quantified for the same form of SS that was used or performed before therapeutic intervention.

Further according to the method, BRT test method data may be acquired at a first location (e.g. a clinical location) and the acquired BRT test method data transferred via electronic means to another location (e.g. a central analysis location) for the herein described analysis and statistical comparisons to be accomplished. The electronic means of data transfer may include, but isn't limited to means of data transfer across a local area network and/or the internet. Consequently, analysis and statistical findings may then be transferred from a central analysis location to a clinical location, where they may be used in various ways by a physician or similarly qualified health care professional for the diagnosis or assessment of central pain.

Further according to the method, BRT test method data may be acquired at a first location (e.g. a clinical location) and the acquired BRT test method data transferred via electronic means to another location (e.g. a central analysis location) for a purpose such as inclusion or increasing the size of various databases of individuals known to be suffering from central pain, individuals known to be suffering from pain that is not central pain, and healthy normal individuals.

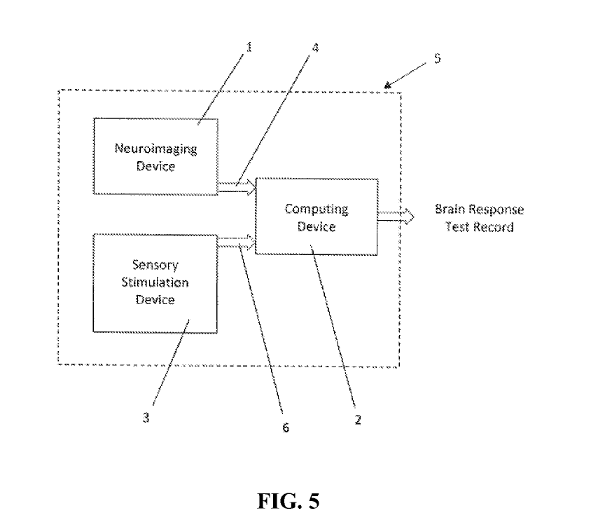

The method of diagnosis and assessment described herein may be accomplished with any number of apparatuses that include apparatuses for providing a neuroimaging test and apparatuses that may be required to create a sensory stimulation. Referring to FIG. 5, a preferred embodiment of an apparatus 5 for diagnosing and assessing central pain may include a neuroimaging device 1 configured to sense and generate images representing central nervous system function, and operably connected to a computing device 2 such as a computer in a way that permits data transfer between the neuroimaging device 1 and the computing device 2. Such connection may be accomplished via a physical cable connection 4 or alternately via a wireless transfer means. The apparatus 5 for diagnosing and assessing central pain may further include a sensory stimulation device 3 configured to stimulate brain activities associated with central sensitivity, and further operably connected to a computing device 2 in a way that permits data transfer between the sensory stimulation device 3 and the computing device 2. Such connection may also be accomplished via a physical cable connection 6 or alternately via a wireless transfer means. In practice, the apparatus 5 for diagnosing and assessing central pain is configured to accomplish the BRT test method described herein. In one embodiment, the computing device 2 may be programmed to operate the neuroimaging device 1 for a period of time, and to collect data, in accordance with the BRT test method, from the neuroimaging device 1 during that time. After such period of time, the computing device 2 is further programmed to suspend data collection from the neuroimaging device 1 and to direct use of a sensory stimulation apparatus 3. Such directing of use may include signaling an operator to manually use a sensory stimulation apparatus 3 on a subject, or may also include programming that automatically controls and operates a sensory stimulation apparatus 3 to create a sensory stimulation on a subject. After use of the sensory stimulation apparatus 3, the computing device 2 may be programmed to further operate the neuroimaging device 1 for another period of time, and to collect additional data in accordance with the BRT test method from the neuroimaging device 1 during that additional period of time. Using neuroimaging device 1 data gathered during both periods of time, the computing device 2 may be further programmed to perform statistical analyses and comparisons on the data in accordance with the BRT test method, and further to transmit data across a network, all according to the method of diagnosis and assessment described herein.

The invention is not limited in any way to the embodiments disclosed herein. In this regard, no attempt is made to show structural details of the disclosed apparatuses or process details of the disclosed methods in more detail than is necessary for a fundamental understanding of the disclosed apparatuses and methods. The description is intended only to make apparent to those skilled in the art how the several forms of the invention may be embodied in practice.

1-52. (canceled)

53. A method for diagnosing and assessing central pain, the method including the steps of:
assessing a subject's brain function using a brain response test (BRT) comprising the steps of:
performing one or more baseline neuroimaging tests,
causing one or more brain responses by applying one or more sensory stimulations, and
performing one or more neuroimaging tests after the step of causing one or more brain responses;
obtaining a quantitative assessment of the subject's brain function; and
making a statistical comparison between the subject's quantitative brain function assessment and one or more databases of quantitative assessments of the brain functions.

54. The method of claim 53 in which the step of making a statistical comparison includes making a statistical comparison between the subject's quantitative brain function assessment and the one or more databases of quantitative assessments of brain functions where the one or more databases include a database of individuals known to be suffering from central pain, a database of individuals known to be suffering from pain that is not central pain, or a database of healthy normal individuals.

55. The method of claim 53 in which the step of applying one or more sensory stimulations includes the application of any noxious, or pain inducing or non-painful means of sensory stimulation causing one or more brain responses.

56. The method of claim 53 in which the step of performing one or more baseline neuroimaging tests includes performing an electroencephalogram (EEG) test.

57. The method of claim 55 in which the step of applying one or more sensory stimulations includes selecting for application one or more sensory stimulations from the group of sensory stimulations consisting of a tender point test, application of mechanical pressure on any part of the body, application of other forms of mechanical stimulation to any part of the body, application of an electrical stimulus, application of a heat-producing stimulus, in vivo or in vitro introduction of a chemical agent, mechanical brushing, controlled physical movements made by the subject, or forms of mental processing such as cognitive exercises.

58. The method of claim 53 in which the step of applying one or more sensory stimulations includes applying a series of sensory stimulations that are made over a period of time in such a way as to produce multiple brain responses.

59. The method of claim 53 in which the step of using a BRT includes assessing changes in brain response over a period of time.

60. The method of claim 53 in which the step of obtaining a quantitative assessment of the subject's brain function includes assessing changes in brain function in response to a sensory stimulation.

61. The method of claim 56 in which performance of an EEG test includes the steps of:
obtaining EEG data for a period of time before the application of a sensory stimulation;
obtaining EEG data and after the application of any one or more sensory stimulations; and
performing a mathematical analysis of obtained EEG data.

62. The method of claim 61 in which each step of obtaining EEG data occurs over a period of time between approximately one second to 15 minutes.

63. The method of claim 53 in which the step of using a BRT test includes the step of quantifying and recording parameters associated with the one or more sensory stimulations.

64. The method of claim 63 in which the step of quantifying and recording parameters occurs at or near the time the subject reports a painful sensation.

65. The method of claim 63 in which the step of quantifying and recording parameters includes quantifying and recording parameters that are selected from a group consisting of location of mechanical pressure, amount of mechanical pressure, parameters of other forms of mechanical stimuli, parameters of forms of electrical stimuli, parameters of forms of heat stimuli, parameters of an introduced chemical agent, parameters of brush strokes, or parameters of a mental exercise.

66. The method of claim 61 in which the performance of an EEG test includes the step of recording EEG for a period of time after application of a final sensory stimulation.

67. The method of claim 66 in which the step of recording EEG for a period of time after application of a final sensory stimulation occurs over a period of time between approximately one second and 15 minutes.

68. The method of claim 66 in which the performance of an EEG test includes the step of producing a resulting EEG data record that includes the brain response test EEG records following each one or more applications of one or more sensory stimulations.

69. The method of claim 68 in which the step of producing a resulting EEG data record includes the step of providing clean EEG data sufficient to perform an EEG analysis, and doing so by extracting non-EEG signals and EEG data acquired during the application of any one or more sensory stimulations from each EEG record.

70. The method of claim 61, in which the step of performing a mathematical analysis is performed on a resulting EEG data record.

71. The method of claim 61, in which the step of performing a mathematical analysis includes the step of selecting one or more analyses from a group consisting of time domain and frequency domain parameters.

72. The method of claim 61, in which the step of performing a mathematical analysis includes the step of selecting one or more analyses from a group consisting of voltage analysis, current analysis, voltage and current analysis, frequency spectrum analysis using Fast Fourier Transform (FFT) analysis, frequency spectrum analysis using a wavelet analysis method, frequency spectrum analysis using absolute power analysis method, frequency spectrum analysis using relative power analysis method, frequency spectrum analysis using phase analysis method, frequency spectrum analysis using coherence analysis method, frequency spectrum analysis using amplitude symmetry analysis method, phase analysis, various forms of network analysis and source localization of electrical activity in the brain using inverse EEG computation analysis.

73. The method of claim 53 in which the step of making a statistical comparison includes the step of performing a mathematical analysis to determine one or more brain measures to support a diagnosis of the presence of one or more brain activities associated with central pain.

74. The method of claim 73, in which the step of performing a mathematical analysis to determine one or more brain measures to support a diagnosis of the presence of one or more brain activities associated with central pain includes the step of analyzing findings from one or more BRT tests.

75. The method of claim 74, in which the step of analyzing findings from one or more BRT tests includes performing one or more analyses to discover one or more brain activities associated with central sensitivity or abnormal brain network connectivity associated with pain processing.

76. The method of claim 74, in which the step of analyzing findings from one or more BRT tests includes the step of statistically comparing findings of a BRT test to BRT test records taken from either healthy normal individuals, individuals suffering from central pain, or individuals that are suffering from pain that is not central pain.

77. The method of claim 74 in which the step of performing a mathematical analysis includes making a determination of EEG abnormalities selected from the group of abnormalities consisting of abnormal levels of EEG power, abnormal levels of coherence between at least two EEG sites, abnormal levels of phase shift between at least two EEG sites, or abnormal levels of EEG relative power in particular regions of the brain.

78. The method of claim 53, in which the method for diagnosing and assessing central pain further includes augmenting assessment of a brain following a BRT by making a physical assessment.

79. The method of claim 74 including the step of diagnosing central pain by:
statistically determining one or more deviations between a subject's one or more BRT measures and like BRT measures obtained from at least one healthy normal individual or at least one individual suffering from pain that is not central pain; then
comparing the one or more deviations to like deviations detected in a sample population of subjects known to be suffering from central pain.

80-88. (canceled)

89. The method of claim 53, in which the step of diagnosing and assessing central pain further includes the step of determining that at least one abnormal measure of the subject's brain associated with central pain corresponds to at least one statistically significant difference finding of a BRT test.

90-97. (canceled)

98. The method of claim 53 in which the step of assessing a subject's brain function further includes the step of making brain measures to identify the presence of one or more brain activities associated with central pain that is a result of central sensitivity.

99. The method of claim 53 in which the step of assessing a subject's brain function further includes the step of making brain measures to identify the presence of one or more brain activities associated with central pain that is a result of abnormal network connectivity.
